(19) United States
(12) Patent Application Publication
(10) Pub. No.: US 2026/0108762 A1
(43) Pub. Date: Apr. 23, 2026

(54) COMPUTER-IMPLEMENTED METHOD FOR MEDICAL GUIDANCE OF A USER IN THE VALIDATION OF A TREATMENT DOSE DISTRIBUTION PROVIDED BY A TREATMENT PLANNING SYSTEM

(71) Applicant: AVATAR MEDICAL, PARIS (FR)

(72) Inventors: Mohamed EL BEHEIRY, PARIS (FR); Elodie BRIENT-LITZLER, PARIS (FR)

(73) Assignee: AVATAR MEDICAL, PARIS (FR)

(21) Appl. No.: 19/146,318

(22) PCT Filed: Jan. 18, 2024
(86) PCT No.: PCT/EP2024/051188

§ 371 Date: Jul. 8, 2025

(30) Foreign Application Priority Data
Jan. 18, 2023 (EP) 23305061.6

(51) Int. Cl. A61N 5/10; A61B 34/00; A61B 34/10; A61N 7/00

(52) U.S. Cl. CPC A61N 5/1039; A61B 34/10; A61B 34/25; A61N 7/00; A61B 2034/107; A61N 2007/0004

(57) ABSTRACT

A computer-implemented method for medical guidance of a user in the validation of a treatment dose distribution provided by a treatment planning system.

### FIELD OF INVENTION

The present invention relates to a computer-implemented method for medical guidance of a user in the validation of a treatment dose distribution provided by a treatment planning system.

### BACKGROUND OF INVENTION

It is known to use a series of images of a subject's body area taken before an intervention. This series of images, called “planning data” or “planning images”, is used by doctors to prepare the intervention, in particular by visualizing the body area on which they must intervene and by locating the diseased tissues as well as determine areas and tissues that are to be preserved. Typically, a three-dimensional model of the body area is computed from the planning images.

In the case of medical interventions where a treatment has to be delivered to a target region of a body subject by the doctors via a probe, precise knowledge of how the probe will interact with the target region is needed, so as to maximize the effects of the treatment while minimizing the risks this treatment may bring.

In the context of radiotherapy or ablation therapy, theoretical three-dimensional treatment dose distributions can be obtained from a treatment planning system. The theoretical three-dimensional treatment dose distributions can be superimposed on a three-dimensional model of the targeted body area computed from planning images.

During the planning phase, it is of help to manipulate a theoretical three-dimensional treatment dose distribution so as to visualize how it will interact with the targeted body area.

However, the current user interfaces available for doctors only display two-dimensional slices, which are difficult to analyze, difficult to manipulate and lack efficient and comfortable visualization properties for the doctors to get intuitive insight on how the theoretical three-dimensional treatment dose distribution will interact with the target body area.

The invention aims at improving the situation.

### SUMMARY

This invention thus relates to a computer-implemented method for medical guidance of a user in the validation of a treatment dose distribution for a subject (e.g., a patient) provided by a treatment planning system, comprising (the following steps):
receiving a body 3D model of at least a (i.e., one) portion of a body of the subject,
receiving treatment plan data representative of a 3D distribution of a treatment dose, to be delivered to a region of the body of the subject during a radiotherapy, an ablation, a laser-based or an ultrasound-based treatment,
generating at least one first cloud of points based on at least a (i.e., one) subset of said treatment plan data, wherein each point is associated with a landmark having displaying characteristics comprising at least (one of the followings:) a shape, a size, an intensity, a transparency, or an orientation, said displaying characteristics being associated with corresponding treatment plan data,
displaying a three-dimensional scene comprising an overlay of the body 3D model and said at least one cloud of points,
   wherein the displaying characteristics of the landmarks associated to said treatment plan data in different depth planes of the three-dimensional scene are distinct, so that said landmarks are better perceived in comparison to classical dose representation of continuous colored overlay.

This allows better visualizing treatment plan data corresponding to different depth planes in the three-dimensional scene, and as a consequence, better visualizing the localization of the treatment plan data. Indeed, the inventors have found that the visibility of an object underneath a cloud of points is better than underneath a continuous coloured layer. Specifically, the inventors have found that the difference in depth of two landmarks associated with different depths in the three-dimensional scene is better visualized with landmarks associated with points of a point cloud than with a continuous coloured layer.

In some embodiments, (the step of) generating said at least one first cloud of points comprises: a) creating a mesh from said treatment plan data, said mesh comprises a plurality of vertices, and wherein, for at least a (i.e., one) subset of said plurality of vertices, each vertex corresponds to a point of said at least one first cloud of points.

In some embodiments, the mesh has a highly symmetrical pattern. For instance, when the treatment plan data is a 3D image composed of a plurality of voxels, the mesh is a cubic crystal system wherein each vertex corresponds to a voxel in the 3D image.

In some embodiments, (the step of) generating the cloud of points further comprises:
b) applying a random coordinate offset to each vertex, said random coordinate offset being smaller than a distance between two neighboring vertices, to obtain offset points, and
c) associating to each offset point a landmark,
   wherein the step of displaying the three-dimensional scene comprises displaying the landmarks at coordinates of the offset points.

Therefore, when the representation of the cloud of points is displayed overlaid on the three-dimensional scene on the display unit, the representation of the cloud of points comprises the offset points. Therefore, the offset points are not aligned as would be points coincident with vertices of the highly symmetrical mesh created from the treatment plan data at step a) but are slightly offset and thus are better visible by a user due to the random offsets.

In an embodiment, one of the displaying characteristics is representative of a dose intensity associated with the treatment plan data.

In some embodiments, for at least one subset of the landmarks, the intensity is variable in time with a predetermined frequency, so as to ease the visualization of said anatomic structures representation belonging to the body 3D model, notably in the region of the body of the subject corresponding to the distribution of the treatment dose.

In some embodiments, the method further comprises, before generating said at least one first cloud of points, receiving a point density parameter, and wherein (the step of) generating said at least one first cloud of points is based on said point density parameter.

In some embodiments, said at least one first cloud of points is dynamic and at least one subset of the landmarks is moving in the vicinity of its initial coordinates, so as to ease the visualization of said anatomic structures representation belonging to the body 3D model, notably in the region of the body of the subject corresponding to the distribution of the treatment dose.

In some embodiments, (the step of) displaying the three-dimensional scene is implemented on a stereoscopic display unit, so as to further increase the perception of the treatment plan data in the 3D space with an enhanced perception of depth.

In some embodiments, the method further comprises receiving at least one interest 3D model of a structure of interest of the subject, and wherein for a first subset of the landmarks, at least one displaying characteristic is assigned to a first value, and for a second subset of the landmarks, said at least one displaying characteristic is assigned to a second value, said second value being different from the first value, wherein the first subset of landmarks is located inside of the at least one interest 3D model, and the second subset of landmarks is located outside of the interest 3D model.

In some embodiments, said structure of interest is representative of an organ at risk.

In some other embodiments, said structure of interest is a target zone.

For instance, the target zone is representative of at least one among gross tumor volume, a clinical target volume, or a planning target volume.

In some embodiments, for each point in the cloud of points, at least one among the displaying characteristics of the corresponding landmark is defined based on a number of beams used to produce the corresponding dose treatment data associated to the point and/or a number of probes to produce the treatment plan data.

In some embodiments, the displaying characteristics associated with corresponding treatment plan data are associated with displaying data being defined as a decreasing function of said 3D distribution treatment dose. In an embodiment, the displaying characteristics are associated to a decreasing function (e.g. inverse function) of the dose intensity and enables the user to identify zones that have not received the treatment.

In some embodiments, the method further comprises (the steps of):
receiving second treatment plan data representative of a second 3D distribution of a second treatment dose, to be delivered to said region of the body of the subject during a radiotherapy or an ablation or a laser-based or an ultrasound-based treatment,
generating a second cloud of points based on at least one subset of said second treatment plan data, wherein each point of the second cloud of points is associated with a landmark having displaying characteristics comprising at least a shape, a size, and an intensity, said displaying characteristics being associated with corresponding second treatment plan data,
displaying an overlay of the three-dimensional scene and said second cloud of points, so that the displayed three-dimensional scene comprises an overlay of the body 3D model, the first cloud of points and second cloud of points.
   In other words, the displaying step consists of displaying the three-dimensional scene further comprising an overlay of said second cloud of points.

In some embodiments, the body 3D model is obtained based on a plurality of images comprising said at least portion of a body of the subject, said plurality of images being CT scan images, MRI images, or cone beam CT images.

In some embodiments, the method further comprises the steps of:
computing at least one 2D projection of said three-dimensional scene on at least one 2D projection plane;
displaying said at least one 2D projection.

In some embodiments, the treatment plan data include information about a type of technology, a type of device, a number of devices, a number of beams, or parameters used in device settings to obtain said treatment plan data.

In some embodiments, a subset of the treatment plan data can be selected through a user interface and the three-dimensional scene displayed in the displaying step comprises an overlay of the body 3D model and of landmarks corresponding to the subset of the treatment plan data.

In some embodiments, said received treatment plan representative of a 3D distribution of a treatment dose is a representation of a treatment 3D distribution. In examples, the receiving of said treatment plan representative of a 3D distribution of a treatment dose comprises (or forms) a computer-implemented method for helping a user in the planification of a treatment to be delivered via a probe to an (effective) target region of a body of a patient (i.e., a subject) by means of a user graphical interface comprising a controller configured to control the interaction between the user and the user graphical interface. said treatment being a radiotherapy, an ablation (also called as ablation therapy), a laser-based or an ultrasound-based treatment.

In such embodiments, the invention more precisely aims at allowing a user (e.g., a medical practitioner) to gain insight on the effects of interaction of a probe delivering a treatment dose to a patient during the planification phase thanks to an interactive method. In other words, the user may first perform the planification of the treatment (in order to obtain/receive a treatment plan data representative of a 3D distribution of a treatment dose) via the user graphical interface. The user may then validate the treatment dose distribution by displaying said overlay of the body 3D model and said at least one first cloud of points.

Said method for helping a user in the planification comprises:
displaying in the user graphical interface a three-dimensional scene comprising a body 3D model of at least a portion of a body of the patient and a probe 3D representation of at least a portion of a probe; said probe 3D representation being interactively controlled by the controller; wherein the three-dimensional scene has a corresponding scene coordinate system attached thereto;
computing a current position and current orientation of the probe 3D representation in the three-dimensional scene coordinate system based on a current position of the mechanical controller in a predetermined user coordinate system;
computing an initial treatment 3D distribution corresponding to said current position and current orientation;
displaying a representation of said initial treatment 3D distribution overlaid with said body 3D model.

This method of helping of planification enables associating a treatment 3D distribution to a position of the controller in the predetermined user coordinate system and to visualize how the treatment distribution is localized with respect to the body 3D model.

This method of helping of planification comprises one or more of the features described in the following embodiments, taken alone or in any possible combination.

In some embodiments, the method further comprises cropping said representation so as to display a 2D cross-section of said initial treatment 3D distribution. Notably, the cropping operation is performed by the method in response to an instruction provided by the user via said controller.

In some embodiments, the initial treatment 3D distribution comprises a set of treatment voxels, each voxel in said set of treatment voxels being associated to a voxel set of parameters.

In some embodiments, for each voxel in the set of treatment voxels, the associated voxel set of parameters comprises an intensity value.

In some embodiments, the three-dimensional scene further comprises a target region.

In some embodiments, the three-dimensional scene further comprises a region of interest.

In some embodiments, the method further comprises identifying target voxels in the set of treatment voxels that are located inside the target region if the three-dimensional scene further comprises a target region, and identifying interest voxels in the set of treatment voxels that are located inside the region of interest if the three-dimensional scene further comprises a region of interest.

In some embodiments, the method further comprises computing a first index of superimposition based on the intensity values of the identified target voxels and displaying said first index of superimposition. Thanks to the first index of superimposition, it is possible to have insight on how the treatment 3D distribution overlaps or fill the target region.

In some embodiments, the method further comprises computing a second index of superimposition based on the intensity values of the identified interest voxels and displaying said second index of superimposition. Thanks to the first index of superimposition, it is possible to have insight on how the treatment 3D distribution overlaps or fill the region of interest.

In some embodiments, the first index of superimposition is a sum of the intensity values of the identified target voxels.

In some embodiments, the second index of superimposition is a sum of the intensity values of the identified interest voxels.

In some embodiments, the first index of superimposition is a first weighted sum of the intensity values of the identified target voxels, the first weighted sum being parameterized by weights, each weight being associated with one among the identified target voxels and being representative of an absorption coefficient of the associated identified target voxel.

In some embodiments, the second index of superimposition is a second weighted sum of the intensity values of the identified interest voxels, said second weighted sum being parameterized by weights, each weight being associated with one among the identified interest voxels and being representative of an absorption coefficient of the associated identified interest voxel.

In some embodiments, the method further comprises:
receiving another current position of the controller in the predetermined user coordinate system,
computing a corrective position and a corrective orientation of the probe 3D representation in the three-dimensional scene coordinate system based on said another current position,
computing a corrective treatment 3D distribution corresponding to the corrective position and corrective orientation,
displaying a representation of the corrective treatment 3D distribution overlaid with said body 3D model.

Therefore, it is possible to explore how a change in the probe position and orientation impact the localization of the treatment 3D distribution with respect to the body 3D model.

In some embodiments, when the three-dimensional scene further comprises a target region, the corrective treatment 3D distribution comprises a set of corrective treatment voxels, and the method further comprises:
identifying corrective target voxels in the set of corrective treatment voxels that are located inside the target region,
computing a first corrective index of superimposition based on the intensity values of the identified corrective target voxels,
displaying the first corrective index of superimposition.

The first corrective index of superimposition helps getting insight on how the corrective position and the corrective orientation of the probe 3D representation has improved or degraded the overlapping or the filling of the target region by the treatment 3D distribution.

In some embodiments, when the three-dimensional scene further comprises a region of interest, the corrective treatment 3D distribution comprises a set of corrective treatment voxels, and the method further comprises:
identifying corrective interest voxels in the set of corrective treatment voxels that are located inside the region of interest,
computing a second corrective index of superimposition based on the intensity values of the identified corrective interest voxels,
displaying the second corrective index of superimposition.

The first corrective index of superimposition helps getting insight on how the corrective position and the corrective orientation of the probe 3D representation has improved or degraded the overlapping or the filling of the region of interest by the treatment 3D distribution.

In some embodiments, the target region is a tumor.

In some embodiments, the region of interest comprises an organ at risk.

In some embodiment, the method further comprises identifying, when the second corrective index of superimposition is higher than a threshold value, displaying an alert message or modifying the display of the region of interest to alert the user.

Indeed, when the region of interest is an organ at risk, the region of interest has to be avoided by the treatment 3D distribution. Therefore, a control of the value of the second index of superimposition helps adjusting or validating a given position and orientation of the probe 3D representation.

In some embodiments, the threshold value is received through the user graphical interface.

In some embodiments, the method further comprises computing angular coordinates representative of an orientation of the probe 3D representation with respect to a reference orientation of said body 3D model.

In some embodiments, the method further comprises displaying said angular coordinates and recording said angular coordinates into an external memory.

In some embodiments, the method further comprises transferring the computed angular coordinates to a mechanical system, mounting the probe, to be used to assist the user in the positioning of the probe during the treatment.

Indeed, when an optimal position and orientation of the probe 3D representation is found, for instance maximizing the first index of superimposition, it may be useful to know and record to which real geometric configuration of the real probe in the real intervention referential this optimal position and orientation of the probe 3D representation corresponds, so as to reproduce it when effectively carrying out the intervention.

In some embodiments, the three-dimensional scene further comprises at least one other probe 3D representation of a portion of another probe, the at least one other probe 3D representation being interactively controlled by the controller and independently from the probe 3D representation, and the method further comprises:
optionally receiving a selection of a subset among the probe 3D representation and the at least one other probe 3D representation in an active state,
computing a complete treatment 3D distribution based on: a) either contributions of the probe 3D representation and the at least one other probe 3D representation, or b) contributions of the received selection,
displaying a representation of the complete treatment 3D distribution overlaid with said body 3D model.

Indeed, in some cases, one probe is not sufficient to deliver enough treatment to the effective target region. Therefore, advantageously, it is possible to interact with a plurality of probe 3D representations and getting insight on their overall effect.

In some embodiments, the complete treatment 3D distribution comprises a complete set of treatment voxels, each voxel in said complete set of treatment voxels being associated to a voxel set of parameters, each voxel set of parameters comprising an intensity value, and the method further comprising:
identifying first complete voxels in the current set of treatment voxels that are located inside the target region,
computing a first complete index of superimposition based on the intensity values of the identified first complete voxels,
displaying said first complete index of superimposition.

The first complete index of superimposition helps getting insight on the effect of all the probes on delivering the treatment to the target region.

In some embodiments, the complete treatment 3D distribution comprises a complete set of treatment voxels, each voxel in said complete set of treatment voxels being associated to a voxel set of parameters, each voxel set of parameters comprising an intensity value, and the method further comprising:
identifying second complete voxels in the complete set of treatment voxels that are located inside the region of interest,
computing a second complete index of superimposition based on the intensity values of the identified second complete voxels,
displaying said second complete index of superimposition.

In some embodiments, the body 3D model is obtained based on a plurality of images comprising said at least portion of a body of the patient, said images being CT scan images, MRI images, or cone beam CT images.

In some embodiments, the user graphical interface comprises a stereoscopic displaying unit.

In some embodiments, the controller is one among a mouse, a 3D controller connected to a virtual reality headset.

In some embodiments, the controller is a home-made controller having a probe-like shape.

Another aspect of the invention pertains to a device comprising a processor, a user graphical interface, said processor being configured to carry out the methods (of guiding and/or planification help) according to any of the embodiments discussed above. In examples, the graphical interface may comprise a controller configured to control said interaction between a user and the user graphical interface.

Another aspect of the invention pertains to a computer program product comprising instructions which, when the program is executed by a computer, cause the computer to carry out the methods (of guiding and/or planification help) according to any of the embodiments discussed above.

The present disclosure further pertains to a non-transitory program storage device, readable by a computer, tangibly embodying a program of instructions executable by the computer to perform a method for helping a user in the planification of a treatment to be delivered via a probe and/or a method for medical guidance of a user in the validation of a treatment dose distribution, compliant with the present disclosure.

Such a non-transitory program storage device can be, without limitation, an electronic, magnetic, optical, electromagnetic, infrared, or semiconductor device, or any suitable combination of the foregoing. It is to be appreciated that the following, while providing more specific examples, is merely an illustrative and not exhaustive listing as readily appreciated by one of ordinary skill in the art: a portable computer diskette, a hard disk, a ROM, an EPROM (Erasable Programmable ROM) or a Flash memory, a portable CD-ROM (Compact-Disc ROM).

### BRIEF DESCRIPTION OF THE DRAWINGS

FIG. 1 is a schematic representation of a device configured to implement the method according to embodiments of the present invention.

FIG. 2 is a schematic representation of a processing unit of the device of FIG. 1.

FIG. 3 is an example of flow chart of the method for medical guidance of a user in the validation of a treatment dose distribution provided by a treatment planning system.

FIG. 4 shows different three-dimensional scenes with an overlay of a body 3D model with a cloud of points, for different points size.

FIG. 5 represents different superpositions of a body 3D model with a cloud of points for different point density values.

FIG. 6 shows an overlay of a cloud of points representative of treatment plan data and segmented organs at risk.

FIG. 7 shows a series of radiographic images with cloud of points representing treatment dose data and corresponding to different threshold conditions.

FIG. 8 shows a comparative of two clouds of points, on the left without an offset of the vertices coordinates and on the right with a random offset of the vertices'coordinates.

FIG. 9 is an example of a three-dimensional scene that can be visualized on a display unit of the device of FIG. 1, comprising a body 3D model, a probe 3D representation and a treatment 3D distribution.

FIG. 10 is an example of flow chart of the method for helping a user in the planification of a treatment to be delivered via a probe to an effective target region of a body of a patient.

FIG. 11 is a first example of a treatment 2D cross section, for a given 2D plane.

FIG. 12 is a second example of a treatment 2D cross section, for a 2D plane different from the given 2D plane of FIG. 5.

FIG. 13 is an example of a three-dimensional scene that can be visualized on a display unit of the device of FIG. 1, the three-dimensional scene comprising a target region.

FIG. 14 is an example of a three-dimensional scene that can be visualized on a display unit of the device of FIG. 1, the three-dimensional scene comprising a target region, a region of interest and a second region of interest.

FIGS. 15-19 show a controller used to move a “virtual ablation probe” which displays and moves an ablation dose represented in cloud of points on a kidney tumour.

### DETAILED DESCRIPTION

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present.

The terms “adapted”, “augmented” and “configured” are used in the present disclosure as broadly encompassing initial configuration, later adaptation or complementation of the present device, or any combination thereof alike, whether effected through material or software means (including firmware).

The term “processing unit” should not be construed to be restricted to hardware capable of executing software, and refers in a general way to a processing device, which can for example include a computer, a microprocessor, an integrated circuit, or a programmable logic device (PLD). The processor may also encompass one or more Graphics Processing Units (GPU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.

It is assumed here that a subject is about to undergo a medical intervention. During this intervention, a portion of his body will be treated by doctors via at least one probe or via at least a beam. For instance, in the case of a radiotherapy intervention, a beam will illuminate the portion of the subject's body. In the case of an ablation therapy, the probe will deliver energy to heat or cool the portion of the subject's body. In the case of an ultrasound-based treatment, ultrasound waves will impinge on the portion of the subject's body.

The invention relates to a computer-implemented method for medical guidance of a user in the validation of a treatment dose distribution provided by a treatment planning system.

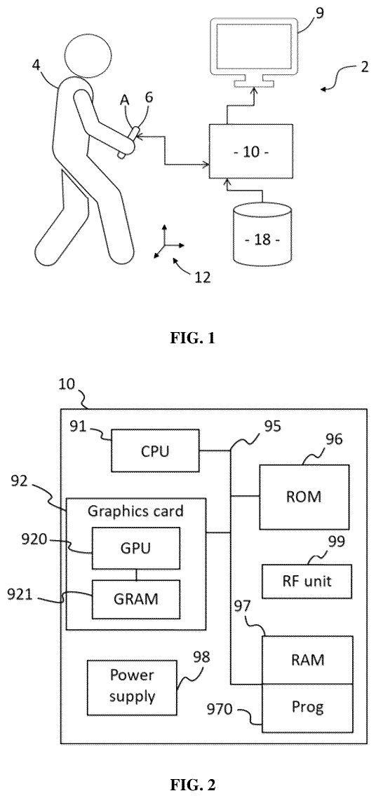

This method may be implemented by a device as illustrated in FIG. 1.

FIG. 1 is a schematic representation of a device configured to implement embodiments of a method for medical guidance of a user in the validation of a treatment dose distribution provided by a treatment planning system.

The device is advantageously an apparatus, or a physical part of an apparatus, designed, configured and/or adapted for performing the mentioned functions and produce the mentioned effects or results. In alternative implementations, the device is embodied as a set of apparatus or physical parts of apparatus, whether grouped in a same machine or in different, possibly remote, machines. The device may for example have functions distributed over a cloud infrastructure and be available to users as a cloud-based service, or have remote functions accessible through an API (“application programming interface”).

The device includes a user graphical interface 2, comprising a controller 6 for interacting with a three-dimensional scene, that will be described more in details below, a display unit 9 for displaying the three-dimensional scene, and a processing unit 10 for processing data received from the controller 6 and for controlling the display unit 9.

### Controller 6

The controller 6 is configured to acquire a current position of a predetermined user point A, of a user 4, in a predetermined user coordinate system 12. The user 4 can be a doctor or any other health professional.

The predetermined user coordinate system 12 is, for instance, fixed with respect to the user's environment. The predetermined user coordinate system 12 uses one or more numbers, or coordinates, to uniquely determine the position of points or other geometric elements in the user's environment.

The controller 6 may include a handheld motion tracking sensor, thereby allowing the user 4 to interact with the three-dimensional scene using hand gestures. In this case, the user point A may be a point of the motion tracking sensor. The three-dimensional scene may be represented in the user graphical interface. More in details, the controller 6 may be equipped with sensors (like accelerometers and gyroscopes) that enable the method to track its position and orientation in 3D space. The method may also relay on the use of external sensors or cameras to enhance tracking accuracy of the controller 6.

The controller 6 may be also equipped with haptic feedback mechanisms. These provide tactile sensations to users, such as vibrations or force feedback, enhancing the sense of touch and interaction within the virtual environment and virtual objects represented in the user graphical interface 2.

The controller 6 may also include other devices, such as a touch screen, a game controller, a mouse and/or a keyboard, thereby further allowing the user 4 to interact with the three-dimensional scene as will be explained later.

The controller 6 may further include buttons and/or switches configured to allow the user 4 to interact with the three-dimensional scene. Such buttons and/or switches may be included in the handheld motion tracking sensor, or may be included in a separate device of the controller 6, such as the touch screen, game controller, mouse and/or keyboard mentioned above.

The controller 6 may include a 3D controller connected to a virtual reality headset. In this case, the user graphical interface 2 and the display unit 9 may be comprised in the virtual reality headset.

The controller may be a home-made controller having a probe-like shape.

Preferably, the controller 6 is configured to allow the user 4 to input (for example through said buttons and/or switches) specific instructions, such as an object display instruction, an object shifting instruction or a transformation instruction. Such instructions advantageously allow the user 4 to add and/or manipulate 3D objects in the three-dimensional scene, as will be described below.

The controller 6 may also be configured to allow the user to manipulate (e.g., to rotate and/or to zoom in on or out of) 3D objects in the three-dimensional scene, and/or to change a direction along which the user 4 views the three-dimensional scene displayed by the display unit 9.

Alternatively, or in addition, the controller 6 includes virtual buttons displayed in the three-dimensional scene to allow the user 4 to interact with the three-dimensional scene, and 3D objects in the three-dimensional scene.

### Display Unit 9

As mentioned previously, the display unit 9 is configured to display the three-dimensional scene, that will be described below.

The display unit 9 may include a screen, as shown on FIG. 1.

Alternatively, and advantageously, the display unit 9 is at least part of a virtual reality headset, thereby allowing stereoscopic visualization of the three-dimensional scene. This is particularly advantageous in the field of medicine, since virtual reality visualization, for example via providing an in-depth visualization, allows the user 4 to have a good understanding of actual volumes and precise localization of objects of interest.

### Processing Unit 10

The processing unit 10 is connected to each of the controller 6 and the display unit 9.

The processing unit 10 corresponds, for example, to a workstation, a laptop, a tablet, a smartphone, programmable logical device (e.g., FPGA) for on-board calculation or a head-mounted display (HMD) such as a virtual reality headset.

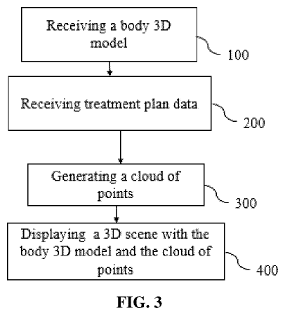

As shown on FIG. 2, the processing unit 10 may comprise the following elements, connected to each other by a bus 95 of addresses and data that also transports a clock signal:
a microprocessor 91 (or CPU);
a graphics card 92 comprising several Graphical Processing Units (or GPUs) 920 and a Graphical Random Access Memory (GRAM) 921; the GPUs are quite suited to image processing due to their highly parallel structure;
a non-volatile memory of ROM type 96;
a RAM 97;
a power supply 98; and
a radiofrequency unit 99.

Alternatively, the power supply 98 is external to the processing unit 10.

The controller 6 is, for instance, connected to at least part of the aforementioned modules, for instance through the bus 95.

The display unit 9 is connected to the graphics card 92, for instance through a suitable interface. For instance, a cable can be used for tethered transmissions, or the RF unit 99 can be used for wireless transmissions.

Each of memories 97 and 921 includes registers, which can designate in each of said memories, a memory zone of low capacity (some binary data) as well as a memory zone of large capacity (enabling a whole program to be stored or all or part of the data representative of data calculated or to be displayed). Also, the registers represented for the RAM 97 and the GRAM 921 can be arranged and constituted in any manner. Each of them does not necessarily correspond to adjacent memory locations and can be distributed otherwise (which covers notably the situation in which one register includes several smaller registers).

When switched-on, the microprocessor 91 loads and executes the instructions of the program 970 contained in the RAM 97 to allow operation of the visualization device 2 in the fashion described in the present disclosure.

As will be understood by a skilled person, the presence of the graphics card 92 is not mandatory, and can be replaced with entire CPU processing and/or other implementations.

### Operation

The device is configured to receive as input 18 a three-dimensional model 3 of the portion of the subject's body. This three-dimensional model 3 is referred to in what follows as body 3D model 3.

The body 3D model 3 can have been preliminary computed based on 2D images of the portion of the subject's body, such as CT scan (“Computed Tomography scan”) images, PET (“Positron Emission Tomography”) images, MRI (“Magnetic Resonance Imaging”) images, or cone beam CT images. Alternatively, the device may be configured to receive as input 18 2D images of the portion of the subject's body, and then compute via the processing unit 10, from the received 2D images, the body 3D model.

The display unit 9 of the user graphical interface 2 is configured to display, to the user 4, a three-dimensional scene comprising the body 3D model 3.

The device is configured to further receive as input 18 treatment plan data representative of a 3D distribution of a treatment dose, to be delivered to a region of the body of the subject during a radiotherapy or an ablation therapy or an ultrasound-based treatment. The processing unit 10 is configured, after reception of the treatment plan data, to generate a cloud of points based on the treatment plan data. Each point in the cloud of point is associated with a landmark having displaying characteristics. Examples of displaying characteristics comprise a shape, a size, an intensity, and a transparency. The displaying characteristics are associated with the corresponding treatment plan data.

In some embodiments, after a cloud of points has been generated, a representation of the cloud of points is displayed overlaid on the three-dimensional scene on the display unit 9. In particular, the representation of the cloud of points is overlaid on the body 3D model 3. This allows better visualizing treatment plan data corresponding to different depth planes in the three-dimensional scene, and as a consequence, better visualizing the localization of the treatment plan data. Indeed, the inventors have found that the visibility of an object underneath a cloud of points is better than underneath a continuous colored layer. In addition, the difference in depth of two landmarks associated with different depths in the three-dimensional scene is better visualized with landmarks associated with points of a point cloud than with a continuous colored layer. Therefore, the use of a cloud of points to visualize the body parts underneath this cloud of points helps a user visualizing in a faster manner those body parts. In that respect, the use of a cloud of points to represent the 3D distribution of a treatment dose contributes to the technical effect of faster visualization of the body parts underneath the cloud of points and of the positioning of treatment plan data over those body parts.

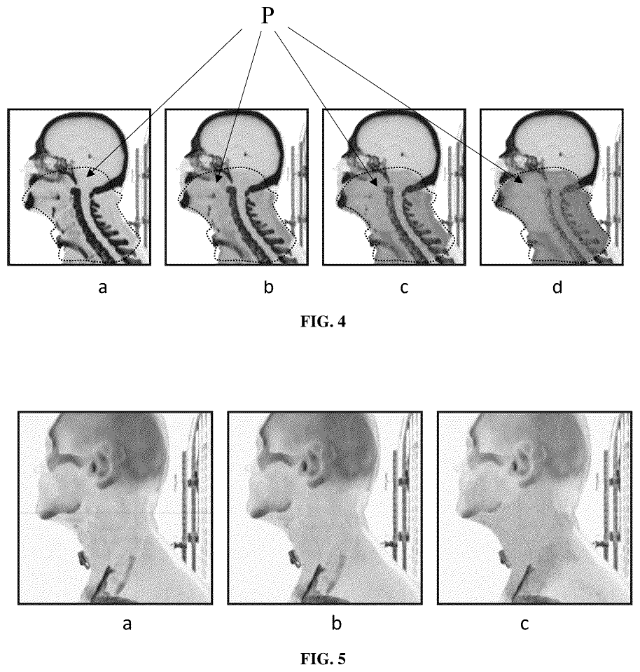

FIG. 4 shows a three-dimensional scene with an overlay of a body 3D model with a cloud of points, for different points sizes. The cloud of points is bounded in region P. From FIG. 4a to FIG. 4d, the point size increases. Therefore, a user can tailor the display of the three-dimensional scene at wish.

Advantageously, the processing unit 10 is configured to generate the cloud of points by:
a) creating a mesh from said treatment plan data, said mesh comprises a plurality of vertices, and wherein each vertex corresponds to a point of the cloud of points,
b) applying a random coordinate offset to each vertex, said random coordinate offset being smaller than a distance between two neighboring vertices, to obtain offset points,
c) associating to each offset point a landmark.

Therefore, when the representation of the cloud of points is displayed overlaid on the three-dimensional scene on the display unit 9, the representation of the cloud of points comprises the offset points. Therefore, the offset points are not aligned as would be points coincident with vertices of a highly symmetrical mesh created from the treatment plan data at step a), but are slight offset and thus are better visible by a user.

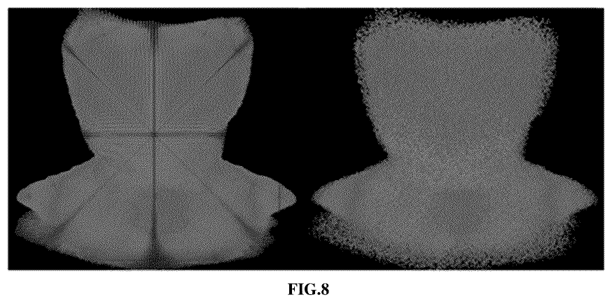

For example, FIG. 8 on the left shows a first cloud of points wherein the points are coincident with vertices of an initial cubical symmetrical mesh, and FIG. 8 on the right shows a second cloud of points wherein the vertices and corresponding points are randomly offset with respect to the initial mesh. Without the offset, one can see artifacts and less accuracy to distinguish between different colors at different depths of the 3D image.

In some embodiments, for at least one subset of the landmarks of the cloud of points, the intensity of the landmarks is not still, and, on the contrary, blinks. In other words, the intensity of the at least one subset of landmarks is variable in time with a predetermined frequency. This further eases the visualization of, for instance, the representation of anatomical structures belonging to the body 3D model and lying underneath the at least one subset of the landmarks.

In some embodiments the display of the cloud of points is dynamic (i.e. not static) and at least one subset of the landmarks is moving (i.e. is displaced) in the vicinity of its initial coordinates, so as to ease the visualization of said anatomic structures representation belonging to the body 3D model, notably in the region of the body of the subject corresponding to the distribution of the treatment dose. The landmark can for instance move (e.g. periodically) around a circle (or other closed trajectories) centered on the coordinates of the vertex it originates from.

In some embodiments, it is possible to set a point density for the cloud of points, before generating it. Varying the point density of the cloud of points allows for customization of the user's visualization settings. In this manner, the user 4 can adjust the point density to his or her convenience.

FIG. 5 illustrates a superposition of a body 3D model and a cloud of points for different point density values. From FIG. 5a to FIG. 5c, the point density parameter increases. Therefore, more points are visible on FIG. 5c than on FIG. 5a.

In some embodiments, the device is configured to receive at least one 3D model of a structure of interest of the subject, referred to as interest 3D model.

For instance, the structure of interest of the subject is one among a gross tumor volume, a clinical target volume, or a planning target volume. The structure of interest is then referred to as a target zone (or equivalently a target region) that has to be reached by the 3D distribution of the treatment dose.

In another example, the structure of interest of the subject is an organ at risk, that has to be avoided by the treatment 3D distribution.

Therefore, in some embodiments where the device receives at least one interest 3D model of a structure of interest of the subject, the cloud of points is divided into two subsets, for instance into a first subset and a second subset. In a first subset of the landmarks located inside of the at least one interest 3D model, at least one displaying characteristic is assigned to a first value. In a second subset of the landmarks located outside of the interest 3D model, said at least one displaying characteristic is assigned to a second value different from the first value. This difference in displaying characteristics allows distinguishing the points inside the structure of interest of the subject from the points outside the structure of interest of the subject. If the structure of interest is one among a gross tumor volume, a clinical target volume, or planning target volume, in this manner, the user 4 can better visualize the effect of the treatment dose inside the structure of interest, and assess if there is a need to optimize this effect. If the structure of interest is an organ at risk, the user 4 can better visualize whether the treatment dose affects the organ at risk, and reacts, for instance to modify the treatment dose.

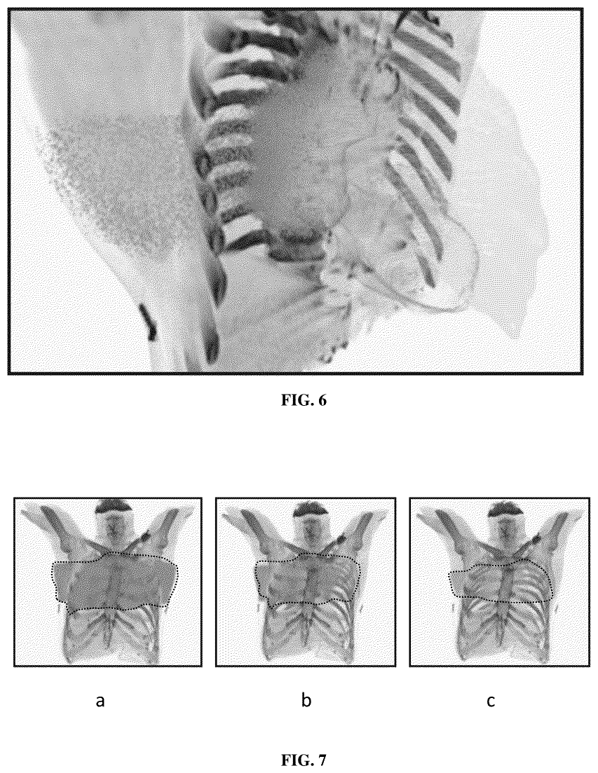

FIG. 6 shows an overlay of a cloud of points representative of treatment plan data and segmented organs at risk.

Advantageously, the treatment plan data are associated with a specific position and orientation of a virtual probe representative of a real probe that would deliver the treatment plan data in real life. Alternatively, the treatment plan data are associated with a specific orientation of a virtual beam representative of a real beam that would deliver the treatment plan data in real life. In this case, it may be possible to vary the position and orientation of the virtual probe, or the virtual beam, with the controller 6. In other words, when the controller 6 is moved in space, in the user's environment comprising the predetermined user coordinate system 12, the position and orientation of the virtual probe is varied accordingly, therefore modifying corresponding coordinates of the treatment plan data with respect to the body 3D model.

Sometimes, the treatment plan data may have been obtained with a plurality of virtual probes (or virtual beams). This is the case for instance when one unique probe, or one unique beam, was not sufficient to deliver enough treatment to a targeted region of the subject's body.

Therefore, in some embodiments, for each point in the cloud of points, at least one among the displaying characteristics of the corresponding landmark is defined based on a number of beams used to produce the corresponding dose treatment data associated to the point and/or a number of probes to produce the treatment plan data. Visualizing the number of beams or probes producing the treatment dose data at a given location of the subject's body allows for adjustment, by the user 4, of the number of beams, or probes, to achieve a given overall treatment dose distribution.

In other embodiments, separate treatment dose data corresponding to separate probes or beams may be received by the device. For instance, the device is configured to receive, in addition to initial treatment dose data, second treatment plan data representative of a second 3D distribution of a second treatment dose, to be delivered to the targeted region of the body of the subject during the medical intervention. In this case, the method further comprises:
generating a second cloud of points based on the second treatment plan data, wherein each point of the second cloud of points is associated with a landmark having displaying characteristics comprising at least a shape, a size, and an intensity, said displaying characteristics being associated with corresponding second treatment plan data,
displaying in overlay of the three-dimensional scene the second cloud of points. In those embodiments, the user can visualize the overall effect of all separate treatment doses and assess the efficiency of this overall effect.

In some embodiments, displaying characteristics associated with corresponding treatment plan data are associated with displaying data being defined as a decreasing function of said 3D distribution treatment dose. For instance, the intensity of the landmarks is a decreasing function of the 3D distribution treatment dose. This helps enhancing the locations in the body 3D model where the treatment dose would not be sufficient and would provoke a reaction of the user 4. Such a reaction could be the manipulation of the controller 6 to displace the cloud of points, or a new computation of treatment dose data with an additional probe or beam.

In some embodiments, the processing unit 10 is configured to compute at least one 2D projection of said three-dimensional scene on at least one 2D projection plane. In this case, the displaying unit is further configured to display at least one 2D projection.

In some embodiments, the treatment plan data include information about a type of technology, a type of device, or parameters used in device settings to obtain said treatment plan data. For instance, the treatment plan data can indicate whether the treatment is an X-ray treatment, a treatment delivered by an electron beam, or by a proton beam. The treatment plan data can also indicate the energy level of the beam used for the treatment. Therefore, one displaying characteristic may pertain to one among such information.

In some embodiments, a subset of the treatment plan data can be selected through a user interface and wherein the three-dimensional scene displayed in the displaying step comprises an overlay of the body 3D model and of landmarks corresponding to the subset of the treatment plan data. This allows for example applying a threshold condition to the treatment plan data and displaying the landmarks corresponding to the treatment plan data satisfying the threshold condition.

FIG. 7 shows a series of radiographic images with cloud of points corresponding to different threshold conditions (from FIG. 7 a to FIG. 7 c, the threshold condition corresponds to a higher dose intensity value. Therefore, less points are visible on FIG. 7 c in contrast to FIG. 7 a). The cloud of points in each of FIG. 7 a to 7 c is bounded in a region defined by a dotted line. For instance, a threshold condition can be input via a user interface by a user. As a consequence, only the points in the cloud of points satisfying the threshold condition can be displayed.

Examples of the receiving of treatment plan data representative of a 3D distribution of a treatment dose (i.e., step 200 in FIG. 3) are now discussed. Such examples in particular discusses receiving said treatment date using a computer-implemented method for helping a user in the planification of a treatment. Such examples may be in combination with any of the embodiments and the examples discussed above.

In such examples, and in reference to FIG. 8, the device may be further configured to receive at least one target region 11, representing a region of the body 3D model 3 (i.e., a volume in the body 3D model 3) that should be treated with the probe by delivering to this target region 11 a desired amount of energy. This target region (one or more) could for example comprise a solid tumor to be treated or any kind of abnormal tissues that should be removed from the patient. The target region 11 may be obtained by a delineation, manually performed by a physician or an automatically performed by a segmentation algorithm. The result of this delineation may than be used to obtain a 3D representation of said target region 11. target region 11 may be as well associated with information to be received as further input to the device such as a thermal conductivity coefficient, a mean, maximum or minimum energy that could be delivered in this region, and the like.

The device may be further configured to receive at least one region of interest 13, representing a region of the body 3D model 3 (i.e., a volume in the body 3D model 3) that should be preserved as much as possible from the exposure to the energy that will be delivered by the probe during the treatment. This(these) region(s) of interest 13 may represent what is generally called an organ at risk. The region of interest 13 may be obtained by a delineation, manually performed by a physician or an automatically performed by a segmentation algorithm. The result of this delineation may than be used to obtain a 3D representation of said region of interest 13. The region of interest 13 may be as well associated with information to be received as further input to the device such as the radio sensitivity of the tissues in the region of interest, a thermal conductivity coefficient, a maximum energy that could be delivered in this region, and the like. This information may be advantageously used during the calculation of the initial 3D treatment distribution.

The device (notably the processing unit 10) is configured to further receive as input 18 a probe 3D representation 5, being a 3D representation of at least a portion of a probe. In the present invention, the probe refers to a device to deliver a treatment, or more generally medical device, that may be used, for example by a physician, to deliver a treatment to a subject. In the present invention such probe/medical device may be, but not limited to, one of the following: an ablation probe (e.g., cryo ablation probe, radiofrequency ablation probe, microwave ablation probe and the like), a radiotherapy gantry, a sealed radiation source for brachytherapy, an ultrasound therapy probe, and the like.

The processing unit 10 may be as well configured to receive a list of predefined information associated to the probe 3D representation 5, such as the type of energy transferred by the probe (e.g., gamma radiation or x-rays radiation in case of radiotherapy, or ultrasound waves in the case of ultrasound therapy probe, etc.), the range of energies that could be delivered and the like. Advantageously, this information may be used by the processing unit 10 for the simulation of the interaction of the energy with the tissues of the body 3D model, during the calculation of the initial 3D treatment distribution. The processing unit 10 could as well be configured to receive other treatment planning information concerning the treatment such as a mean energy to be transferred or a maximum energy to not overcome to preserve healthy tissues.

The processing unit 10 may be as well configured to receive a list of predefined information associated to the probe 3D representation 5, such as the type of energy transferred by the probe (e.g., gamma radiation or x-rays radiation in case of radiotherapy, or ultrasound waves in the case of ultrasound therapy probe, etc.), the range of energies that could be delivered and the like. Advantageously, this information may be used by the processing unit 10 for the simulation of the interaction of the energy with the tissues of the body 3D model, during the calculation of the initial 3D treatment distribution. The processing unit 10 could as well be configured to receive other treatment planning information concerning the treatment such as a mean energy to be transferred or a maximum energy to not overcome to preserve healthy tissues.

As described above, the display unit 9 of the user graphical interface 2 is configured to display, to the user 4, a three-dimensional scene comprising the body 3D model 3 and the probe 3D representation 5 of the at least one portion of a probe. The three-dimensional scene has a corresponding scene coordinate system attached thereto. There is a correspondence between the predetermined user coordinate system 12 and the corresponding scene coordinate system.

FIG. 9 is an example of a three-dimensional scene comprising the body model 3 and the probe 3D representation 5, as displayed on the display unit 9 in FIG. 1. The cloud of points is not shown in FIG. 9 and only the iso-surface lines are shown.

In some embodiments, the probe 3D representation 5 is interactively controlled by the controller 6. Indeed, the controller 6 is configured to allow the user to interact with the objects represented in the scene (i.e., virtual environment), such as the probe 3D representation 5 and the body 3D model 3. As a consequence, when the controller 6 is moved in space by the user 4, in the user's environment comprising (i.e., associated to) the predetermined user coordinate system 12, the probe 3D representation 5 moves in the corresponding scene coordinate system. To a position and orientation of the controller 6 in the predetermined user coordinate system 12, corresponds a position and an orientation of the probe 3D representation 5 in the scene coordinate system.

When the user 4 manipulates the controller 6, the position and spatial orientation of the controller 6 is changed. These changes in position and spatial orientation of the controller 6 are continuously monitored by the controller sensors (e.g., IMU embedded in the controller 6) and are translated from the user coordinate system 12 into the scene coordinate system. The person skilled in the art knows different technics allowing transforming real positions of a controller into a virtual environment. As a consequence, the position and orientation of the probe 3D representation 5 change in the scene coordinate system.

For a given position and orientation of the probe 3D representation 5 in the scene coordinate system, a corresponding treatment 3D distribution 7 may be computed. This calculated treatment 3D distribution comprises a set of treatment voxels. The processing unit may be configured to calculate for each voxel of said set of treatment voxels a set of parameters, that will be called in the present description “voxel set of parameters”.

Said initial treatment 3D distribution may be calculated, not only on the based on the given position and orientation of the probe 3D representation 5 in the scene coordinate system, but also on the base of the type of energy delivered/transferred by the probe that is simulated. Indeed, as explained above, the probe to be simulated in the present invention may be chosen from a list of multiple probes/medical devices and be configured to deliver a treatment based on the delivery of different type of energy. Depending in the type of energy to be transferred to the tissues, the physical interactions between the “carrier” of this energy (i.e., radiation, microwaves, ultrasounds, etc.) and the tissues change and so it changes the 3D distribution of the energy (i.e., treatment 3D distribution) in the body 3D volume. Advantageously, the information on the type of probe (i.e., on the type of energy delivered by the probe) allows the processing unit 10 to calculate the initial 3D distribution on the base of the simulation of the corresponding physical interactions. Furthermore, the other information concerning the treatment to be delivered by the probe may be taken into account during the calculation such as the list of predefined information associated to the probe 3D representation 5, the information associated with the target region 11 and/or the information associated with the region of interest 13.

One of the parameters (of said voxel set of parameters) that could be calculated for each voxel is an intensity value representative of an amount of energy transferred by the probe to the voxel. In the case of radiation therapy or brachytherapy, other parameters that may be calculated such as the dose delivered to the tissue associated to the voxel, calculated in Gray, or the effective dose, calculated in Sieverts, if the potential biological effects of ionization radiation and the radio sensitivity of the tissues is taken into account. In the case of cryo ablation, laser ablation and the like, a parameter calculated may be the temperature, or the maximum temperature, that will be reached in the voxel during the treatment.

For instance, in the case of a radiotherapy intervention, the probe (e.g., a gantry) is configured to deliver a beam that will illuminate (i.e., irradiate) the portion of the patient's body, notably the totality of the target region 11 while trying to avoid as much as possible the region(s) of interest 13 (i.e., organs at risk). The corresponding initial treatment 3D distribution 7 then corresponds to a set of voxels, the voxels being referred to as treatment voxels. The set of treatment voxels is representative of the spatial distribution of the energy that is transferred by the beam delivered by the probe in a given position and orientation. Thus, the set of treatment voxels covers a 3D area (i.e., a volume) in the scene coordinate space. The 3D area is computed at least based on the probe real characteristics (i.e., list of predefined information associated to the probe 3D representation 5) and on the probe 3D representation position and orientation in the scene coordinate space. In addition, each treatment voxel in the set of treatment voxels is associated to a voxel set of parameters. For instance, for a given treatment voxel, the voxel set of parameters comprises an intensity value, representing the dose that would be delivered by the radiation beam to the tissue associated to the voxel. Each intensity value is computed based on the probe real characteristics (i.e., list of predefined information associated to the probe 3D representation 5) and on the probe 3D representation position and orientation in the scene coordinate space. The treatment planning information may be used as additional information for a more accurate calculation of the intensity values.

Similarly, in the case of an ablation therapy, the probe delivers/transfers energy that will heat or cool the portion of the patient's body. The initial treatment 3D distribution 7 corresponding to the delivered energy can be represented by a set of voxels. In other words, the initial treatment 3D distribution 7 represents all the voxels of the body 3D model 3 that will have a change in temperature because they will be affected by the energy transfer, either by improving the energy of the biological system when heat is transferred (i.e., absorbed) or by reducing the energy of the biological system when the heat is extracted (i.e., cooling down).

In the case of an ultrasound-based treatment, the probe emits ultrasound waves that will impinge on the portion of the patient's body. The treatment 3D distribution 7 corresponding to the emitted ultrasound waves can be represented by a set of voxels. In other words, in the case of ultrasounds, the treatment 3D distribution 7 comprises all the voxels of the body 3D model 3 that are reached by the ultrasound waves emitted by the probe. The ultrasound waves may be used in physical therapy to apply controlled amounts of energy to targeted tissues for therapeutic benefits. This can include promoting tissue healing, reducing inflammation, and providing pain relief. The energy is delivered through the skin using a specialized ultrasound transducer (i.e., ultrasound probe). As consequence, the treatment 3D distribution 7 provides a representation of the energy that would be transferred by the ultrasound waves to the tissues when the ultrasound probe is positioned in a given position and orientation with respect to the body 3D model 3.

In the case of a laser-based treatment, the probe emits a laser that will illuminate the portion of the patient's body. As consequence, in this case, the treatment 3D distribution 7 provides a representation of the energy that would be transferred by the laser to the tissues when the probe is positioned in a given position and orientation with respect to the body 3D model 3.

As described according to all the example hereabove, the treatment 3D distribution 7 provides a representation of the energy that would be transferred to the tissues represented in the body 3D model 3. Therefore, the treatment 3D distribution 7 may be as well called 3D energy distribution.

In some embodiments, after that for one given position and orientation of the probe 3D representation 5 in the scene coordinate system, a corresponding treatment 3D distribution 7 has been computed, a representation of the computed treatment 3D distribution 7 is displayed overlaid on the three-dimensional scene on the display unit 9. An example of such a representation is visible on FIG. 9. Notably in FIG. 9, the probe 3D representation 5 and a visual representation of the treatment 3D distribution 7 are represented in the three-dimensional scene (i.e., virtual environment) at the same time that a partial representation of the body 3D model 3. In this example, the visual representation of the treatment 3D distribution 7 allow to easily identify the distal voxels/external boundaries of the volume of the treatment 3D distribution 7. In this way the user will advantageously have the information at once of the position and direction of the probe 3D representation 5 with respect to the body 3D model 3 and that all voxels comprised in this visual representation of the treatment 3D distribution 7 will be affected by the transferred of energy applied with the probe. Thanks to the overlapping of the visual representation of the treatment 3D distribution 7 with a section of the body 3D model 3, the user is also able to visualize which structures of the patient will be affected during the treatment, when the probe is positioned according to his/her choice.

Furthermore, the section of the body 3D model 3 or the structure comprised in the body 3D model 3 (i.e., vascular system, bones, etc.) to be visualized may be choose during the visualization by the user via the interaction with the controller 6. For example, the user graphical interface 2 may be configured to implement an interactive mean called cropper, allowing to virtually crop the volumes represented in the virtual environment. Finally, the overlay of the probe 3D representation with the body 3D model enables the user to optimize the placement of the probe as relative to specific anatomical structures. For stick-shaped probes, such as notably ablation probes, this overlay may help the user to select an entry point and angle (i.e., spatial orientation) of the probe which might avoid critical organs or vessels.

In one embodiment, the device is configured to receive and store a selection, from the user, of one position and spatial orientation of the probe 3D representation 5 with respect to the body 3D model 3, as additional treatment planning information. This additional treatment planning information may be integrated into a treatment plan, which may comprise a list of at least one treatment action to be performed by the user (i.e., surgeon) with the probe on the patient during the treatment. Each additional treatment planning information selected by the user may be integrated into the treatment plan as one treatment action to be performed and be optionally also associated with an order for its execution. In other word, the user may determine a treatment plan with one or more actions to be performed in an order that he/she selected. In one example, the probe may be mounted on a robot (i.e., mechanical system) and the treatment plan may be used to control said robot to execute the treatment action(s) under the supervision of the user.

In one embodiment, the device is configured to provide as output, for example visual output, said treatment plan.

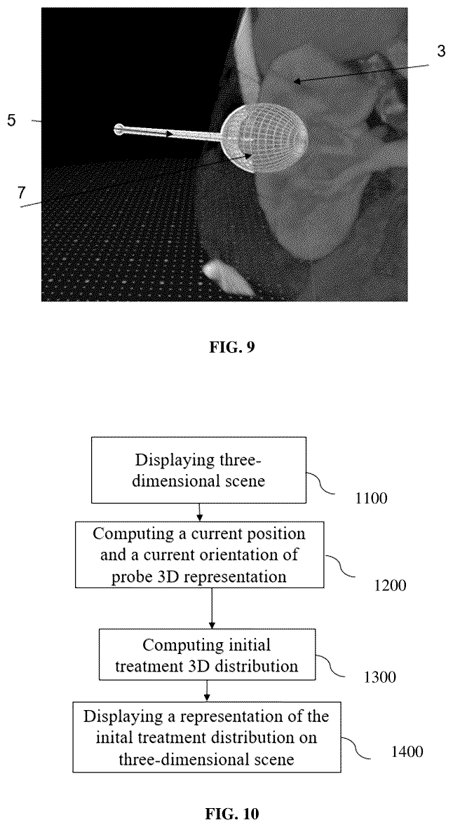

FIG. 10 is an example of flow chart of the method for helping the user 4 in the planification of the treatment to be delivered via the probe to the effective target region of the body of the patient.

In a step 1100, the three-dimensional scene is displayed in the display unit 9 of the user graphical interface 2. The three-dimensional scene comprises the body 3D model 3 and the probe 3D representation 5.

In a step 1200, a current position and current orientation of the probe 3D representation 5 in the three-dimensional scene coordinate system is computed based on a current position and spatial orientation of the controller 6 in the predetermined user coordinate system 12.

In a step 1300, an initial treatment 3D distribution 7 corresponding to said current position and current orientation is computed.

In a step 1400, a representation of the initial treatment 3D distribution 7 is displayed in the display unit 9, overlaid with the body 3D model 3.

In some embodiments, the user graphical interface 2 comprises croppers.

Via the croppers, the user 4 may crop the body 3D model relative to a 2D plane in the scene coordinate system, so that a 2D cross section of the body 3D model, referred to as body 2D cross section, is displayed on the visualization unit. For instance, the body 2D cross section results from an intersection between the body 3D model and the 2D plane.

Alternatively, via the croppers, the user 4 may crop, relative to a 2D plane in the scene coordinate system, the representation of the treatment 3D distribution computed for a given position and orientation of the probe 3D representation in the scene coordinate system, so that a 2D cross section of the treatment 3D distribution, referred to as treatment 2D cross section, is displayed on the display unit 9. For instance, the treatment 2D cross section results from an intersection between the treatment 3D distribution 7 and the 2D plane.

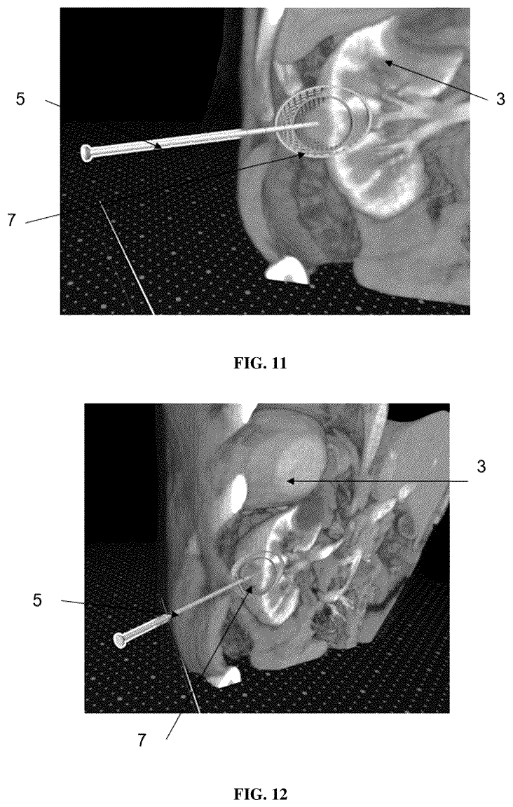

FIGS. 11 and 12 are two examples of a treatment 2D cross section, for different 2D planes. The cloud of points is not shown in FIG. 11 and FIG. 12; only the iso-surface lines are shown.

In some embodiments, the three-dimensional scene further comprises a target region 11. The target region 11 is a digital representation of the effective target region of the body of the patient to which the treatment needs to be delivered. The target region 11 may be representative of a region that needs to be reached or hit by the treatment while manipulating the probe during the intervention. For instance, the target region 11 is a tumor to reduce or eliminate. Such a target region 11 may be visible on the body 3D model 3, when the body 3D model 3 has been computed from preoperative images. FIG. 13 shows an example of the three-dimensional scene comprising the target region 11. A label of the target region 11 can be displayed. On FIG. 13, the target region 11 is a tumor. FIG. 13 does not show the cloud of points and only the iso-surface lines are shown.

When the three-dimensional scene comprises such a target region 11, the method for helping the planification of the treatment further includes a step of identifying voxels in the three-dimensional scene, referred to as target voxels, that are both located in the target region and belong to the set of treatment voxels, computed for a given position and orientation of the probe 3D representation 5 in the scene coordinate system. In other words, the step of identifying allows the user getting an insight about how, for the given position and orientation of the probe 3D representation 5, the treatment 3D distribution 7 overlaps or fills the target region 11.

As an indicator of how the treatment 3D distribution 7 fills the target region, a first index of superimposition I₁ can be computed. The first index of superimposition I₁ can be displayed on the three-dimensional scene, as visible on FIG. 13. When for a given treatment voxel, the voxel set of parameters comprises an intensity value, the first index of superimposition I₁ can be computed based on the intensity values of the identified target voxels. Advantageously, this first index of superimposition I₁ provides a quantitative information on the percentage of the target region that would be affected by the energy of the calculated treatment 3D distribution 7.

In one example, the first index of superimposition I₁ is the sum of the intensity values of the identified target voxels. Alternatively, the first index of superimposition I₁ may be calculated as the sum or the weighted sum of the intensity values of x % lower intensity voxels among the identified interest voxels, x ranging from approximately 1 to 20. This is an important information for the user, notably when cancerous tissues must be eradicated, to ensure that with the currently calculated treatment 3D distribution 7 a minimum amount of energy, sufficient to destroy the target cells, is transferred to the target region.

In another example, the first index of superimposition I₁ is a first weighted sum of the intensity values of the identified target voxels. The first weighted sum can be parameterized by weights, where each weight is associated with one among the identified target voxels and is representative of an absorption coefficient of the associated identified target voxel. This example is for instance relevant when it is desired to take into account the different transfer properties of different tissues in the target region.

In some embodiments, the three-dimensional scene further comprises a region of interest 13. The region of interest 13 may be representative of an organ at risk, and thus needs to be protected from the treatment while manipulating the probe during the intervention. In other words, the region of interest 13 may be representative of areas to be avoided by the treatment. Such a region of interest 13 may be visible on the body 3D model 3, when the body 3D model 3 has been computed from preoperative images.

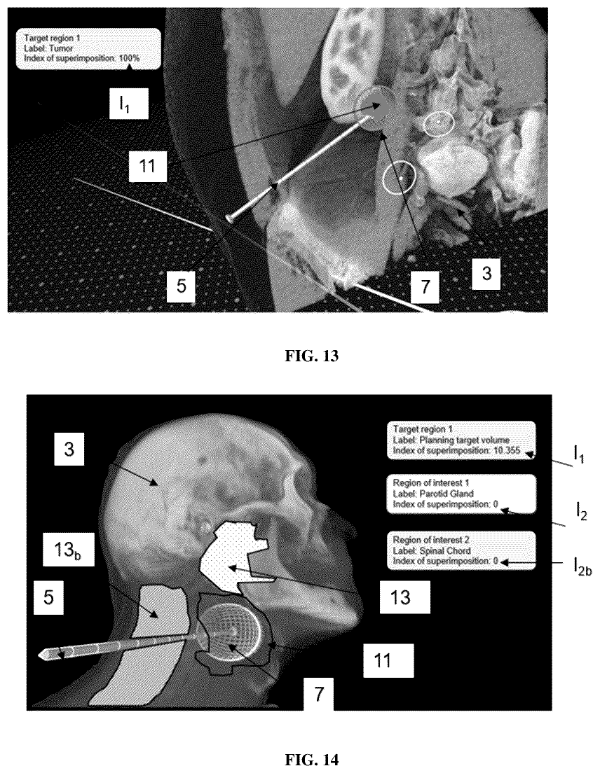

FIG. 14 is an example of a three-dimensional scene comprising a target region 11 and a region of interest 13. A label of the target region 11 and a label of the region of interest 13 are displayed. On FIG. 14, the target region 11 is a planning target volume and the region of interest 13 is a parotid gland of the patient. The three-dimensional scene may further comprise other regions of interest. On FIG. 14, a second region of interest 13b is visible and a label of the second region of interest 13b is displayed. On FIG. 14, the region of interest is a spinal cord. FIG. 14 does not show the cloud of points and only the iso-surface lines are shown.

When the three-dimensional scene comprises such a region of interest 13, the method for helping the planification of the treatment further includes a step of identifying voxels in the three-dimensional scene, referred to as interest voxels, that are both located in the region of interest and belong to the set of treatment voxels, computed for a given position and orientation of the probe 3D representation 5 in the scene coordinate system. In other words, the step of identifying allows the user getting an insight about how, for the given position and orientation of the probe 3D representation 5, the treatment 3D distribution 7 overlaps or hits the region of interest.

As an indicator of how the treatment 3D distribution hits the region of interest, a second index of superimposition I₂ can be computed. The second index of superimposition I₂ can be displayed on the three-dimensional scene, as visible on FIG. 14. When for a given treatment voxel, the voxel set of parameters comprises an intensity value, the second index of superimposition I₂ can be computed based on the intensity values of the identified interest voxels.

In one example, the second index of superimposition I₂ is the sum of the intensity values of the identified interest voxels. The second index of superimposition I₂ may be calculated as sum or weighted sum of the Y % voxels with the highest intensity values among the identified interest voxels, where Y may range from approximately 1 to 20.

In another example, the second index of superimposition I₂ is a second weighted sum of the intensity values of the identified target voxels. The second weighted sum can be parameterized by weights, where each weight is associated with one among the identified interest voxels and is representative of an absorption coefficient of the associated identified interest voxel. This example is for instance relevant when it is desired to take into account the different absorption (i.e., transfer) properties of different tissues in the region of interest.

When the region of interest is representative of areas to be avoided by the treatment, the second index of superimposition I₂ can be used to adjust the position and orientation of the probe 3D representation 5. For instance, a threshold value can be set as a maximum value that cannot be exceed. When the second index of superimposition I₂ is higher than the threshold value, an alert message can be displayed. Alternatively, the display of the region of interest may be modified to alert the user that the overlap between the treatment, for the current position and orientation of the probe, is excessive and might even be dangerous. For instance, the threshold value is inputted by the user through the user graphical interface.

In some embodiments, as already mentioned, the three-dimensional scene comprises both a target region 11 and a region of interest 13. Therefore, both the first index of superimposition I₁ and the second index of superimposition I₂ can be displayed, as illustrated on FIG. 14.

When the three-dimensional scene comprises other regions of interest, such as on FIG. 14, the corresponding second indexes of superimposition can be displayed. On FIG. 14, another second index of superimposition I₂ b is displayed and corresponds to the second region of interest 13b.

The method of the present invention may be configured to follow the movement of the controller 6, held by the user, in a continuous manner. As consequence, the current position and spatial orientation of the controller 6 are continuously received and used to update in real time the probe 3D representation 5 in the virtual environment and as well calculate and display in real time the corresponding treatment 3D dose distribution.

In some embodiments, the user can interact with the user graphical interface 2 through the controller 6, in order to, starting from a starting position and a starting orientation of the probe 3D representation, modify the position and orientation of the probe 3D representation 5, and, as a consequence, to modify the treatment 3D distribution 7. Therefore, in those embodiments, the method further comprises:
receiving another current position of the controller 6 in the predetermined user coordinate system, following an interaction of the user with the controller 6,
computing a corrective position and a corrective orientation of the probe 3D representation 5 in the three-dimensional scene coordinate system based on said another current position,
computing a corrective treatment 3D distribution corresponding to the corrective position and corrective orientation,
displaying a representation of the corrective treatment 3D distribution overlaid with said body 3D model.

The corrective treatment 3D distribution corresponds to a new set of voxels, the voxels in the new set of voxels being referred to as corrective treatment voxels.

When the three-dimensional scene comprises a target region 11, the method for helping the planification of the treatment further includes a step of identifying voxels in the three-dimensional scene, referred to as corrective target voxels, that are both located in the target region 11 and belong to the set of corrective treatment voxels, computed for the corrective position and corrective orientation of the probe 3D representation 5 in the scene coordinate system.

As an indicator, the first index of superimposition I₁ is updated, now referred to as first corrective index of superimposition, and computed in a similar way to the first index of superimposition previously described. The first corrective index of superimposition can be displayed on the display unit 9.

When the three-dimensional scene comprises a region of interest 13, the method for helping the planification of the treatment further includes a step of identifying voxels in the three-dimensional scene, referred to as corrective interest voxels, that are both located in the region of interest 13 and belong to the set of corrective treatment voxels, computed for the corrective position and corrective orientation of the probe 3D representation 5 in the scene coordinate system.

As an indicator, the second index of superimposition I₂ is updated, now referred to as second corrective index of superimposition, and computed in a similar way to the second index of superimposition previously described. The second corrective index of superimposition can be displayed on the display unit 9.

Advantageously, a convenient position and orientation of the probe 3D representation 5 can be found when interacting with the user graphical interface 2. Such a convenient position and orientation corresponds for instance to a maximum value of the first index of superimposition I₁ when the three-dimensional scene comprises a target region 11. Therefore, in some embodiments, the method for helping the user in the planification of the treatment to be delivered via the probe further includes computing angular coordinates representative of an orientation of the probe 3D representation 5 with respect to a reference orientation of said body 3D model 3. The angular coordinates will thus be usable to reproduce and reposition the real probe during the real medical intervention aiming at delivering the treatment to the effective target region of the body of the patient in the same manner as during the interaction with the user graphical interface 2. Conveniently, the angular coordinates can be displayed on the display unit 9 and recorded into an external memory in view of the real medical intervention. The computed angular coordinates may be transferred to a mechanical system, mounting the probe, to be used to assist the user in the positioning of the probe during the treatment.

Sometimes, it may occur that one unique probe is not sufficient to deliver enough treatment to the target region. In this case, using a second probe can be envisaged.

Therefore, in some embodiments, the three-dimensional scene further comprises at least one other probe 3D representation, representative of at least a portion of another probe. The at least one other probe representation is, like the probe 3D representation 5, interactively controlled by the controller 6. For instance, the number of probe 3D representations in the three-dimensional scene is N, with N being an integer higher or equal to 2.

In those embodiments, each probe 3D representation might be manipulated and moved to a current position and a current orientation, which are recorded. A corresponding individual treatment 3D distribution corresponds to each of the current position and current orientation. A complete treatment 3D distribution can then be computed based on each corresponding individual treatment 3D distribution.

For instance, each individual treatment 3D distribution corresponds to an individual set of voxels, referred to as treatment voxels. Each treatment voxel is associated with an intensity value. The complete treatment 3D distribution can then correspond to a set of voxels, referred to as complete set of treatment voxels, where each voxel in the complete set of treatment voxels is associated with a total intensity value. For example, the total intensity value is computed as a sum of the intensity values of each individual treatment 3D distribution at this voxel.

In those embodiments, when the three-dimensional scene comprises a target region 11, the method for helping the planification of the treatment further includes a step of identifying voxels in the three-dimensional scene, referred to as first complete voxels, that are both located in the target region 11 and belong to the complete set of treatment voxels.

As an indicator, the first index of superimposition I₁ is updated, now referred to as first complete index of superimposition, and computed in a similar way to the first index of superimposition I₁ previously described. The first complete index of superimposition can be displayed on the display unit 9 of the user graphical interface 2.

In those embodiments, when the three-dimensional scene comprises a region of interest 13, the method for helping the planification of the treatment further includes a step of identifying voxels in the three-dimensional scene, referred to as second complete voxels, that are both located in the region of interest 13 and belong to the complete set of treatment voxels.

As an indicator, the second index of superimposition I₂ is updated, now referred to as second complete index of superimposition, and computed in a similar way to the second index of superimposition I₂ previously described. The second complete index of superimposition can be displayed on the user graphical interface 2.

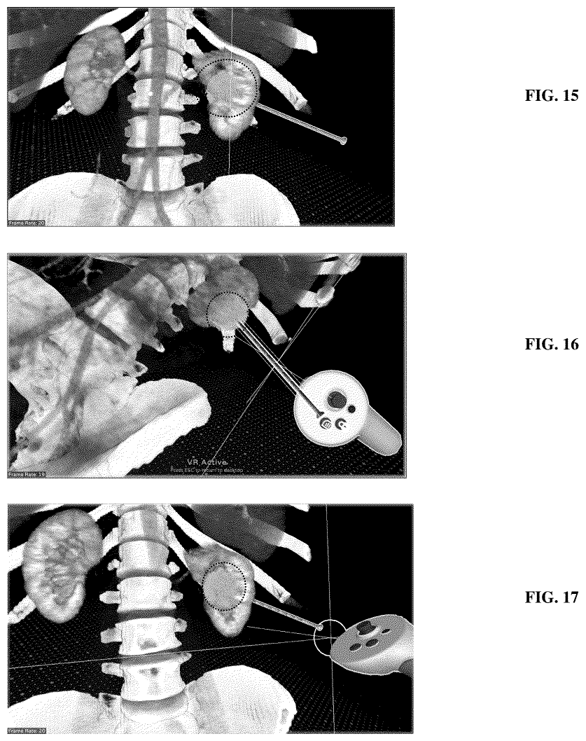

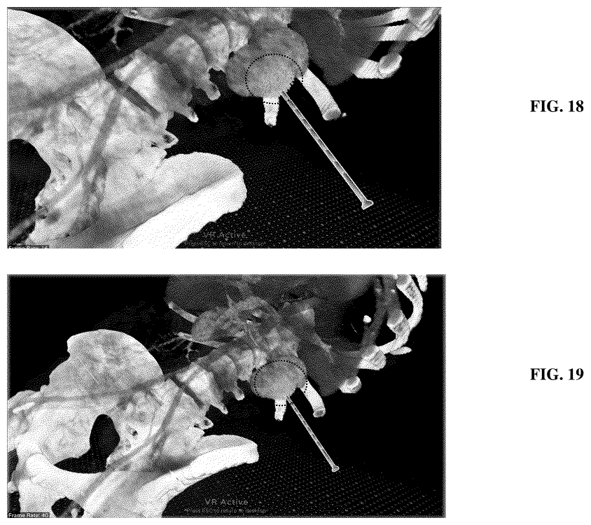

FIGS. 15-20 show a controller used to move a “virtual ablation probe” which displays and moves an ablation dose represented in cloud of points on a kidney tumour in an image obtained by CT scan. The cloud of points in each of FIGS. 15 to 19 is bounded in a region defined by a dotted line.

A person skilled in the art will readily appreciate that various embodiments disclosed may be combined without departing from the scope of the invention. Of course, the present invention is not limited to the embodiments described above as examples. It extends to other variants.

1-15. (canceled)

16. A computer-implemented method for medical guidance of a user in the validation of a treatment dose distribution for a subject provided by a treatment planning system, comprising:
receiving a body 3D model of at least one portion of a body of the subject,
receiving treatment plan data representative of a 3D distribution of a treatment dose, to be delivered to a region of the body of the subject during a radiotherapy, an ablation, a laser-based or an ultrasound-based treatment,
generating at least one first cloud of points based on at least one subset of said treatment plan data, wherein each point is associated with a landmark having displaying characteristics comprising at least a shape, a size, an intensity, a transparency, or an orientation, said displaying characteristics being associated with corresponding treatment plan data,
displaying a three-dimensional scene comprising an overlay of the body 3D model and said at least one first cloud of points,
wherein the displaying characteristics of the landmarks associated to said treatment plan data in different depth planes of the three-dimensional scene are distinct, so that said landmarks are better perceived in comparison to classical dose representation of continuous colored overlays.

17. The method according to claim 16, wherein generating said at least one first cloud of points comprises:
a) creating a mesh from said treatment plan data, said mesh comprises a plurality of vertices, and wherein, for at least one subset of said plurality of vertices, each vertex corresponds to a point of said at least one first cloud of points.

18. The method according to claim 17, further comprising:
b) applying a random coordinate offset to each vertex, said random coordinate offset being smaller than a distance between two neighboring vertices, to obtain offset points, and
c) associating to each offset point a landmark,
wherein displaying the three-dimensional scene comprises displaying the landmarks at coordinates of the offset points.

19. The method according to claim 16, wherein one of the displaying characteristics is representative of a dose intensity associated with the treatment plan data.

20. The method according to claim 16, wherein for at least one subset of the landmarks, the intensity is variable in time with a predetermined frequency, so as to ease the visualization of said anatomic structures representation belonging to the body 3D model, notably in the region of the body of the subject corresponding to the distribution of the treatment dose.

21. The method according to claim 16, further comprising, before generating said at least one first cloud of points, receiving a point density parameter, and wherein the generating said at least one first cloud of points is based on said point density parameter.

22. The method according to claim 16, wherein said at least one first cloud of points is dynamic and at least one subset of the landmarks is moving in the vicinity of its initial coordinates, so as to ease the visualization of said anatomic structures representation belonging to the body 3D model, notably in the region of the body of the subject corresponding to the distribution of the treatment dose.

23. The method according to claim 16, wherein displaying the three-dimensional scene is implemented on a stereoscopic display unit, so as to further increase the perception of the treatment plan data in the 3D space with an enhanced perception of depth.

24. The method according to claim 16, further comprising receiving at least one interest 3D model of a structure of interest of the subject, and wherein for a first subset of the landmarks, at least one displaying characteristic is assigned to a first value, and for a second subset of the landmarks, said at least one displaying characteristic is assigned to a second value, said second value being different from the first value, wherein the first subset of landmarks is located inside of the at least one interest 3D model, and the second subset of landmarks is located outside of the interest 3D model.

25. The method according to claim 24, wherein said structure of interest is representative of an organ at risk.

26. The method according to claim 24, wherein said structure of interest is a target zone.

27. The method according to claim 26, wherein the target zone is representative of at least one among gross tumor volume, a clinical target volume, or a planning target volume.

28. The method according to claim 16, further comprising:
receiving second treatment plan data representative of a second 3D distribution of a second treatment dose, to be delivered to said region of the body of the subject during a radiotherapy or an ablation or a laser-based or an ultrasound-based treatment,
generating a second cloud of points based on at least one subset of said second treatment plan data, wherein each point of the second cloud of points is associated with a landmark having displaying characteristics comprising at least a shape, a size, and an intensity, said displaying characteristics being associated with corresponding second treatment plan data,
displaying the three-dimensional scene further comprising an overlay of said second cloud of points.

29. The method according to claim 16, wherein said body 3D model is obtained based on a plurality of images comprising said at least one portion of a body of the subject, said plurality of images being CT scan images, MRI images, or cone beam CT images.

30. The method according to claim 16, further comprising:
computing at least one 2D projection of said three-dimensional scene on at least one 2D projection plane;
displaying said at least one 2D projection.

31. The method according to claim 16, wherein the treatment plan data include information about a type of technology, a type of device, a number of devices, a number of beams, or parameters used in device settings to obtain said treatment plan data.
